(19) United States
(12) Patent Application Publication
(10) Pub. No.: US 2021/0121150 A1
(43) Pub. Date: Apr. 29, 2021

(54) ON-BOARD CHARGED PARTICLE THERAPY COMPUTED TOMOGRAPHY SYSTEM

(71) Applicant: WILLIAM BEAUMONT HOSPITAL, Royal Oak, MI (US)

(72) Inventors: Xuanfeng DING, Bloomfield Hills, MI (US); Jun ZHOU, Troy, MI (US); Xiaoqiang LI, Troy, MI (US); Yan DI, Auburn Hills, MI (US); Peyman KABOLIZADEH, Royal Oak, MI (US); Craig STEVENS, Tampa, FL (US)

(73) Assignee: WILLIAM BEAUMONT HOSPITAL, Royal Oak, MI (US)

(21) Appl. No.: 17/258,761

(22) PCT Filed: Jul. 8, 2019
(86) PCT No.: PCT/US2019/040793

§ 371 Date: Jan. 8, 2021

Related U.S. Application Data
(60) Provisional application No. 62/695,554, filed on Jul. 9, 2018.

(51) Int. Cl. A61B 6/00

(52) U.S. Cl. CPC A61B 6/54; A61B 6/482; A61B 6/4435; A61B 6/4241

(57) ABSTRACT

An on-board proton imaging system may include a continuous rotation gantry configured to generate proton beams during rotation thereof to penetrate a patient object, a beam detector arranged opposite of the gantry around the object and configured to receive residual proton beams having passed through the object, and a controller in communication with the gantry and a multilayer detector. The controller may be configured to instruct the gantry to generate the proton beams based on patient factors, receive data from the detector indicating at least an energy level of the residual beams, and generate a three-dimensional image based on the received data.

### CROSS-REFERENCE TO RELATED APPLICATIONS

This application claims the benefit of U.S. Provisional Application No. 62/695,554 filed Jul. 9, 2018, the disclosure of which is hereby incorporated in its entirety by reference herein.

### TECHNICAL FIELD

Disclosed herein are on-board charged particle therapy computed tomography systems.

### BACKGROUND

Current proton imaging systems and designs may use single particle tracking methods for proton energy acquisition and reconstruction. The current design is limited to passive scattering gantry nozzle 2D imaging projection per static gantry angle.

### SUMMARY

An on-board proton imaging system may include a continuous rotation gantry configured to generate proton beams during rotation thereof to penetrate a patient object, a beam detector arranged opposite of the gantry around the object and configured to receive residual proton beams having passed through the object, and a controller in communication with the gantry and a multilayer detector. The controller may be configured to instruct the gantry to generate the proton beams based on patient factors, receive data from the detector indicating at least an energy level of the residual beams, and generate a three-dimensional image based on the received data.

A proton imaging system may include a memory configured to store patient factors and a controller in communication with the memory and configured to instruct a continuous rotation gantry to generate proton beams based on the patient factors to penetrate a patient object, receive proton beam data from a beam detector indicating at least an energy level of residual beams having passed through the object, and generate a three-dimensional image based on the received data.

### BRIEF DESCRIPTION OF THE DRAWINGS

The embodiments of the present disclosure are pointed out with particularity in the appended claims. However, other features of the various embodiments will become more apparent and will be best understood by referring to the following detailed description in conjunction with the accompanying drawings in which:

FIG. 1 illustrates an example on-board pCT system;

FIG. 2 illustrates a side view of the example on-board pCT system of FIG. 1;

FIG. 3 illustrates an example system having a multilayer flat panel pixelated proton residual energy detector;

FIG. 4 illustrates an example system having a ring-shaped multilayer pixelated proton residual energy detector;

FIG. 5 illustrates a detailed view of the gantry of the on-board pCT system;

FIG. 6 illustrates an example proton spot pattern including fluence, position, scanning sequence (partial delivery), and spot energy modulation at various gantry and detector rotation positions due to the continuous gantry rotation and imaging;

FIG. 7 illustrates an example diagram of residual proton energy;

FIG. 8 illustrates an example proton pencil beam spot measurement acquired by the multilayer residual energy detector using spot decomposition method;

FIG. 9 illustrates an example fluence on each layer of the detector;

FIG. 10 illustrates an example spectrum of proton residual energy on each sub-spot across different detector layers;

FIG. 11a illustrates an example image where a proton spot with small number of protons are detected by the detector;

FIG. 11b illustrates an example image where a proton spot with large number of protons are detected by the detector;

FIG. 11c illustrates an example image of a statistically computed residual energy on the sub-spots; and

FIG. 12 illustrates an example process for the on-board pCT system.

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

Proton imaging may use pencil beam technology to acquire patient images. Currently, proton imaging has been used to acquire traditional 2D projection based proton computed tomography (pCT) reconstruction with matured pCT imaging detectors and rotational passive scattering gantry. However, these designs may be incompatible with pencil beam scanning (PBS) technology, require an extra bulky system, be expensive, and only be capable of passive-scattering with 2D projections, leading to slow imaging acquisition.

The current design is limited to passive scattering gantry nozzle with a very low dose rate, so the system is able to count each individual particle when it passes through the entrance detector and exist detector. The current system only acquires 2D imaging projection per static gantry angle. It requires up to four position sensitive detectors (PSD) before the patient and a set of PSD after the patient. A residual energy range detector (RERD) is used to detect the energy spectrum. Due to the high particle current and intensity of current PBS technique which dominates the proton therapy market, the existing proton imaging system is not able to handle numerous particles at the same time, as a result, significant modifications of the current PBS charged particle therapy system is needed in order to acquire the proton imaging with the existing technique.

The existing single particle tracking methods take hours in order to acquire sufficient 2D proton imaging and reconstruct into 3D. Such time-consuming technique is not clinically feasible, nor has any commercial valuable. Due to its single proton tracking method, the efficiency is slow. It is not efficient to be implemented on a PBS clinical machine. A pixelated multilayer residual energy range detector (RERD) is used to derive energy spectrum of a proton spot.

However, in this disclosure, the existing charged particle pencil beam scanning (PBS) gantry nozzle can be used directly for the proton imaging acquisition where traditional ionization chamber strips are normally used. No major modification is needed. In other words, PBS high current and fluence can be used directly or compatibly in this technique for 3D charged particle imaging reconstruction. In addition, the 3D charged particle imaging acquisition, post-processes and reconstruction can be finished in several minutes, which will have significant clinical and commercial values.

Disclosed herein is an on-board proton imaging system that acquires proton imaging using an on-board pCT gantry, with the capability to simultaneously acquire x-ray images. The disclosed system includes a proton imaging pixelated residual energy detector consisting of multilayer ionization chambers, multilayer CMOS detector, or multilayers of scintillator detectors. The residual proton beams received at the various layers of the detector may be used to generate a 3D image.

The system allows the manufacture to reduce the thickness, weight and the cost of the proton imaging panel that could be installed on a particle therapy gantry, or on-board charged particle computed tomography. This system further provides the methods and system which is able to acquire, post-process and reconstruct the 3D proton images through a continuously rotational pencil beam scanning (PBS) charged particle therapy gantry directly which is compatible with the state-of-art charged particle therapy system.

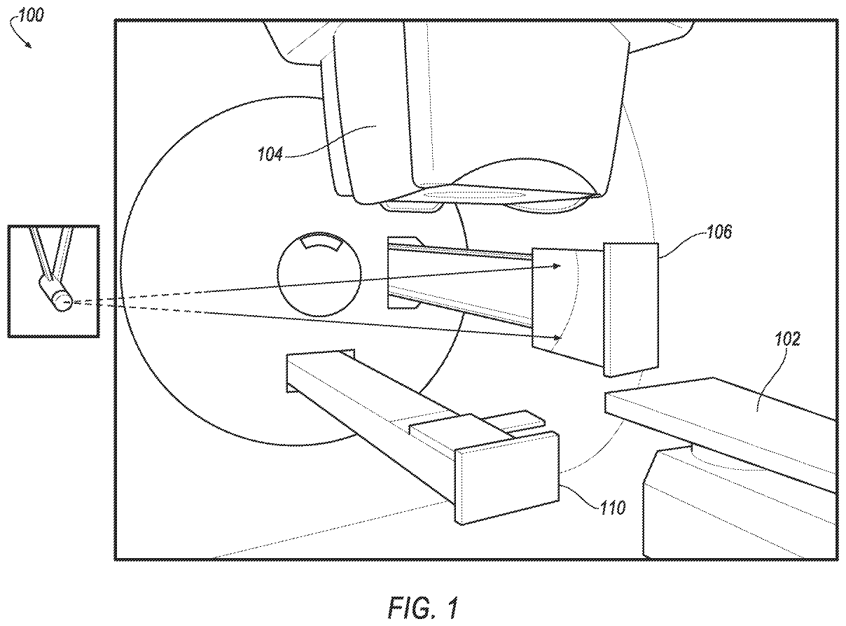

FIG. 1 illustrates an example on-board pCT system 100. The on-board pCT system may include a typical computed tomography (CT) imaging system. The system 100 may include a patient table 102 configured to receive the patient and allow the patient to lay vertically during the imaging procedure. A chair, couch, or recliner may also be provided for the patient's comfort in lieu of a table. The system 100 may include a gantry 104 configured to rotate around the patient table 102 during treatment or imaging. The system 100 also includes a cone-beam computed tomography (CBCT)/kV X-ray panel detector 106 configured to acquire imaging relating to the same.

The system 100 may also include a proton energy detector 110. The proton energy detector 110 may be an imaging panel including multilayer ionization chambers, multilayer CMOS detectors, or multilayer scintillator detectors. The detector 110 may be arranged generally opposite the gantry and be configured to receive proton pencil beams from the gantry 104.

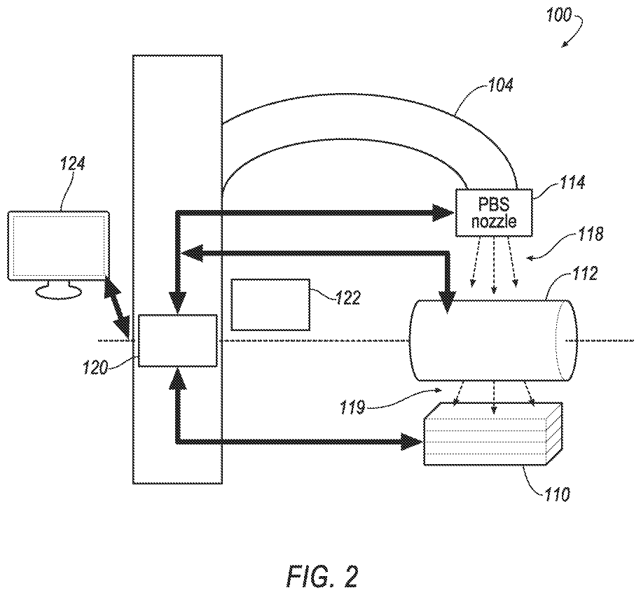

FIG. 2 illustrates a side view of the example on-board pCT system 100 of FIG. 1. The system 100 includes the gantry 104 which may include a pencil beam scanning (PBS) gantry nozzle 114. The nozzle 114 may be an accelerometer configured to produce particle beams 118. The particle beam 118 may extend from the nozzle 114, project to the patient table, and be received at the multilayer pixelated residual energy detector 110.

A controller 120 may control the system 100, including the gantry 104, the nozzle 114, and the multilayer pixelated residual energy detector 110. The controller 120 may be generally coupled to memory 122 for operation of instructions to execute equations and methods described herein. In general, the controller 16 is programmed to execute the various methods as noted herein. The controller 120 may include the models described herein. For example, the controller 120 may generate a sequence of proton beam generation and image acquisition. The sequence may create a continuous rotational gantry on-board pCT based on pencil beam techniques. The sequence may include instructions for the gantry 104 and nozzle 114 to emit particle beams 118 of various spot sizes, energy, and angles.

The controller 120 may generate the sequence based on various known factors or “pre-knowledge” acquired from previous imaging scans taken of the particular patient. Such factors or data may be acquired from the memory 122 or input at a monitor 124. The previous imaging may include previous CT scans, MRIs, X-rays, PETs, ultrasounds, etc.

As explained, the proton energy detector 110 may be an imaging panel including multilayer ionization chambers, multilayer CMOS detector, multilayer scintillator detectors, or other forms of multilayer detectors. The detector 110 may be configured to receive residual particle beams 118 from the gantry 104. A portion of these beams 18 may extend through the patient and into the energy detector 110. The detector may have one or multiple layers 115 with a 2D pixelated detector. The majority of the particles will stop in the multilayer detector so that the range of the particles will be derived. That is, this residual beam 119 may include the particles or energy left over after passing through the object 112. The residual beam (also referred to herein as residual particles 119) may stop at one of the various layers 115 of the energy detector 110, indicating a proton energy of the respective residual beam 118 and the spot position on the imaging pixelated panel.

All the layers 115 may work simultaneously, and the detectors may work in an integration mode or pulse mode (to provide temporal information). The detectors may use direct energy/dose collection, similar to ion chambers, or indirect mode in which the radiation is converted to light or other forms of data. The respective proton energy of the residual beam 119 is received by the controller 120 and used by the controller 120 to iteratively and continuously reconstruct an image based on the proton energy. The detector pixels may be binned so that a spot covered by multiple pixels can be grouped into sub-spots and processed accordingly.

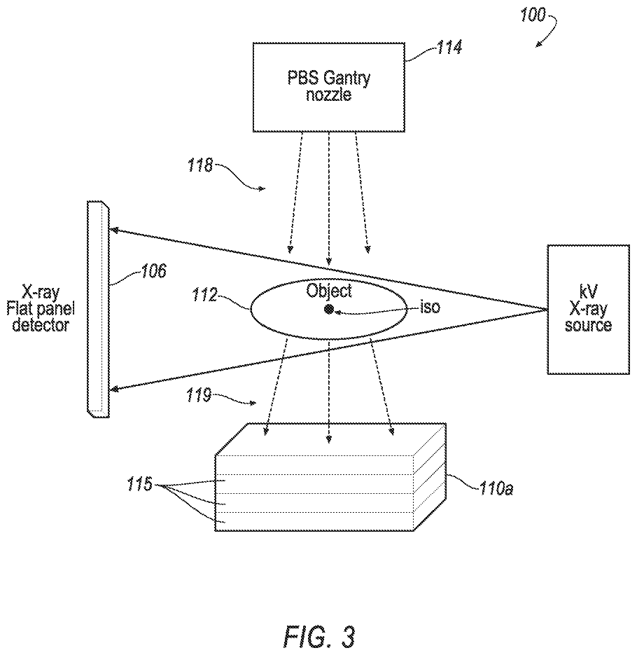

FIG. 3 illustrates an example system 100 having a flat multilayer pixelated proton residual energy detector 110a. The flat multilayer energy detector 110a may include a plurality of layers 115. As explained above, each layer may be configured to receive the residual particles from the residual beam 119. The detector 110a may then transmit a particle location and layer to the controller 120. Each layer may be associated with a corresponding energy level. That is, if the residual beam 119 penetrates and stops at a layer, the residual energy of the beam 119 may be determined. The location may include coordinates indicating the relative spot of each residual beam 119 along the specific detector layer 115.

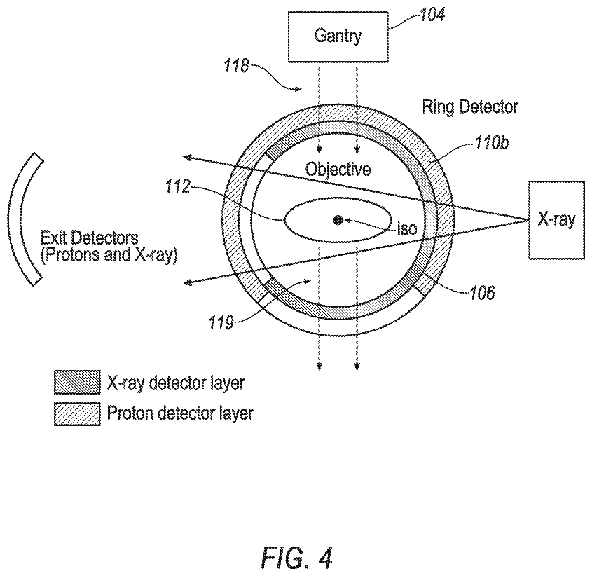

FIG. 4 illustrates an example system 100 having a ring-shaped multilayer pixelated proton residual energy detector 110b. The ring-shaped detector 110b may be configured to surround the object 112, at least in part. While flat and ring-shaped panels are illustrated, other shapes and configurations may also be appreciated. The x-ray panel detector 106 may be similarly shaped, as shown in the Figures.

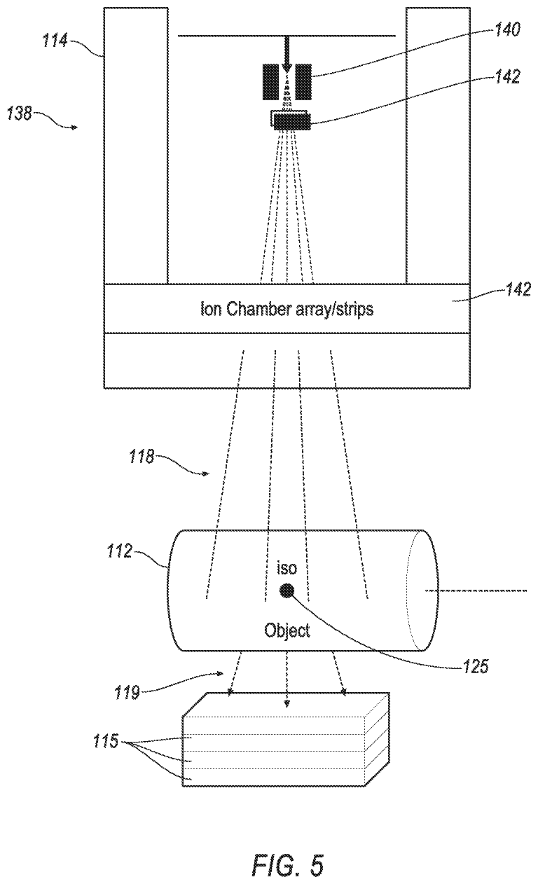

FIG. 5 illustrates a detailed view of the gantry 104. The isocentric gantry 104 is configured to continuously rotate along the isocenter 125. Additionally or alternatively, the patient table 102 (as shown illustrated FIG. 1), or other form of chair, may continuously rotate along the isocenter 125. The gantry 104 may include the nozzle 114 and an energy layer system 138. The energy layer system 138 may be configured to generate the beams 118. The layer system 138 may include two sets of scanning magnets 140. Each set of scanning magnets 140 may include a pair of x and y scanning magnets which is perpendicular to the particle beam path or direction. The scanning magnets 140 are configured to steer the particle beam 118 in X and Y direction, forming a pencil beam spot position, direction and spot scanning sequence.

The layer system 138 may further include an ionization chamber 142 configured to receive the beams from the magnets 140. The ionization chamber 142 measures and records the particle beam's fluences, positions, and directions. This proton beam data may be used by the controller 120 to further generate the three-dimensional image. The layer system 138 may further include degraders, beamline magnets, etc., configured to select the appropriate energy levels and transfer the particle beams from an accelerator. The nozzle 114 then produces the particle beams 118 for transmission to the iso. The energy layer system 138, including the scanning magnets 140 and ionization chamber 142, provides initial particle beam information before entering the patient's body such as particle beam's energy, (fluence, position and directions.

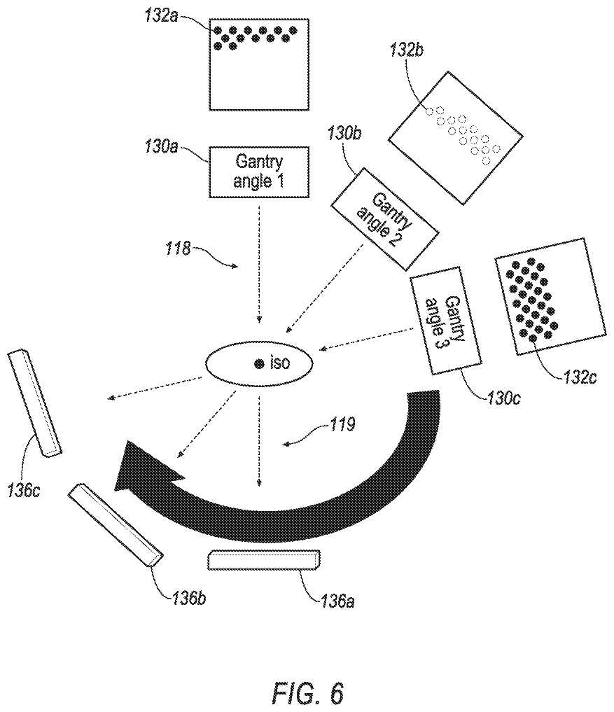

FIG. 6 illustrates charged particle spot patterns including charge particle spot's fluence, position, and scanning sequence (partial delivery) and spot energy modulation at various gantry 104 and detector 110 rotational angles. Each gantry position or a plurality of gantry control point may generate particle beams of certain energy, fluoresce or different position and directions. For example, at a first position 130a, the particle beams may have a first spot pattern/spot scanning sequence 132a, a first fluoresce, a first energy and first particle beam position and directions. At a second position 130b, the charge particle beams may have a second spot pattern 132b and a second fluoresce, a 2nd energy and 2nd particle beam positions and directions. In one example, the proton beam may use the high energy spots for the first spot pattern 132a to scan a high Water Equivalent Path Length (WEPL) region. At the following gantry angle, e.g., second position 130b, the PBS spots may switch to medium energy to scan the median WEPL region. In the last position, e.g. third position 130c, the PBS spots will switch to low energy to scan the low WEPL region. If not all the spots are able to be delivered at a specific angle, the remaining spots may be delivered at the next adjacent gantry angle.

At each gantry position, the gantry 104 may generate particle beams 118 of different energy, fluoresce or position and direction. For example, in the same gantry control point, the particle beams may have a spot pattern, with different fluoresce in different spot position and direction.

Thus, the sequence, as defined by the controller 120, may generate proton beams 118 of varying energies and fluoresces at various angles and positions. The energy detector 110 may rotate with the gantry 104 at respective first 136a, second 136b, and third 136c positions and acquire the residual charged particles 119. The gantry 104 may continuously rotate while the nozzle 114 that produces charged particle beams. The detector 110 acquires residual proton beams 119 and provides the same to the controller 120 (not shown in FIG. 4). Each of the charged particle beams at the first, second and third positions 132a, 132b, 132c may have differing spot patterns (position and direction or scanning sequence in x, y coordinate) and fluoresce, as well as initial proton energy.

Based on these partial projection images at each gantry angle, an iterative image reconstruction system is applied to regenerate the image based on the residual proton beam received at the detector 110.

Assuming the residual energy (K) of the protons reaching detector pixels is Gaussian distributed:

[symbol]

(K)= [symbol] (σ,K₀·r·f)

where K₀ is the initial energy of the protons, r E R represents the most likely path for the protons reach a spot on the detector, and f is a vector represents the relative stopping power on each voxel of the object.

For each measured residual energy on a spot, the probability

$$P ( K = k ) = \frac{1}{\sqrt{2 π} σ} {e}^{- \frac{1}{2} {( k - ɛ )}^{2 / {σ}^{2}}} , with ϵ = {K}_{0} \cdot r \cdot f$$

Assuming a total of M projection angles are used; and at each projection angle i∈M, the maximum number of spots (covering the whole detector area) is N. In a continuous delivery and partial scanning/reconstruction scenario,

εᵢⱼ=k₀·r_{i,j}·f

where projection angle i=1 to M, and j<=N at each angle i.

Thus, the likelihood for all measured signals is:

$$P ( K = k , f ) = {\prod }_{i , j} P ( {K}_{i , j} = {k}_{i , j} )$$

and the reconstruction problem is to find the stopping power map f that maximizes L:

$$L ( f ) = - {\sum }_{i , j} {( \frac{{k}_{i , j} - {ɛ}_{i , j}}{{σ}_{i , j}} )}^{2} - β R ( f )$$

where βR(f) is used to penalize the roughness.

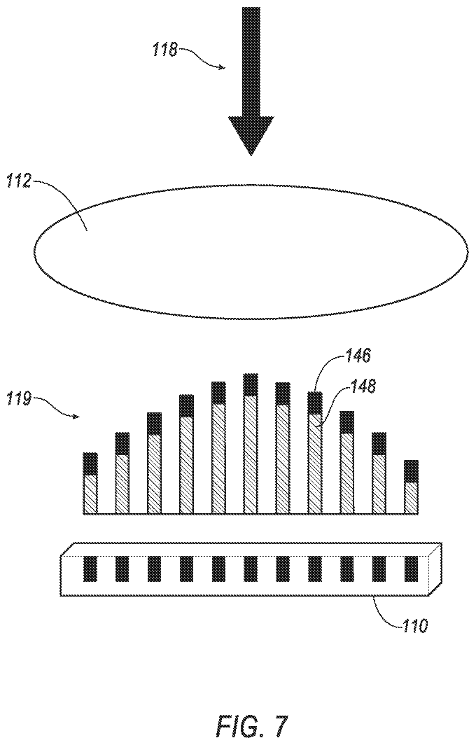

FIG. 7 illustrates an example diagram of residual proton energy. Given pre-knowledge P (CT Hounsfield Unit from simulation CT or CBCT) of the object being imaged, a 2D projection Wᵢ of water equivalent path length (WEPL) is calculated for projection angle i. A 1D gradient calculation is conducted on both u and v directions (u and v are two orthogonal directions on the image plane) so that the high gradient regions can be identified. High gradient regions will be scanned with less spot spacing. The 2D projection Wᵢ is divided into L, sub-regions based on their WEPL values. Charged particle spot scanning energy for each sub-region will be optimized so that the proton energies in the exiting spots are within the detector capture range. The residual beam 119 may include a first water equivalent thickness 146 that is proportional to the particle energy, as well as a second water equivalent thickness 148 that is additional energy to allow the particles to pass through the object 112 and reach the detector 110.

In other words, the thickness 146 is the energy difference between the initial energy of the proton beam emitted from the gantry and a minimum energy required to penetrate the patient body at that location. The residual energy received in the detector 110 indicates this difference. The initial energy is programmed by the controller 105 based on the patient body geometer or patient factors. The initial energies for each beam are higher than the minimum energy so that the proton may penetrate the patient's body and reach the detector 110.

The proton beam energy WEPL range may be represented by Rmin to Rmax (for example 4 cm to 50 cm), and the detector WEPL range may be represented by D0 to Dmax (here, D0 is the thickness of an initial filter to remove unwanted protons with low energies, and Dmax is the max WEPL thickness of the detector). Further, the WEPL projection (at a specific angle) may be calculated from the pre-knowledge/patient factors and has range PWEPL of 0 to Pmax. A total of N energy layers be used, with any energy Rᵢ=Rₘᵢₙ+i×ΔR, i∈[0, N] and R_{N≤}5 Rₘₐₓ. One of the choice of ΔR=(Rₘₐₓ−Rₘᵢₙ)/N and ΔR<(Dₘₐₓ−D₀).

A lookup table may be created for any spots on the pre-calculated WEPL projection P(x, y), the corresponding proton energy could be selected based on the following equation:

$${R}_{i} = {R}_{min} + i \times Δ R$$

$$i = ⌊ \frac{P ( x , y ) - {D}_{0}}{Δ R} ⌋$$

in which └ ┘ represents a floor operation, i≥0.

An example range of stopping points can be found at https://www.nist.gov/pml/stopping-power-range-tables-electrons-protons-and-helium-ions.

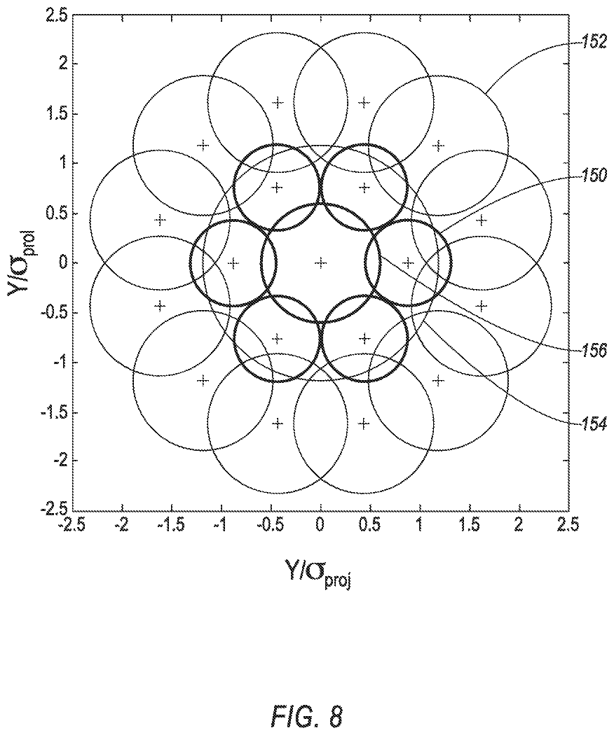

FIG. 8 illustrates an example sub-spot measurement generated by the energy detector 110 where each layer of the detector 110 is configured to receive a residual proton beam 119 and generate sub-spots by dividing these residual proton beams 119 into sub-distributions of N. In the example shown in FIG. 5a, N=19. The sub-spots may include first concentric circles 150 and second concentrate circles 152. The sub-spots may include a certain amount of first concentric circles 1, and a certain amount of second concentric circles J. In the example shown in FIG. 5a, 1=6 and J−12. FIG. 7 illustrates proton pencil beam spot 154 measured by the multilayer residual energy detector. Sub-spots (e.g., first concentric circles 150, second concentric circles 152, and a third circle 156) are then generated by spot decomposition method.

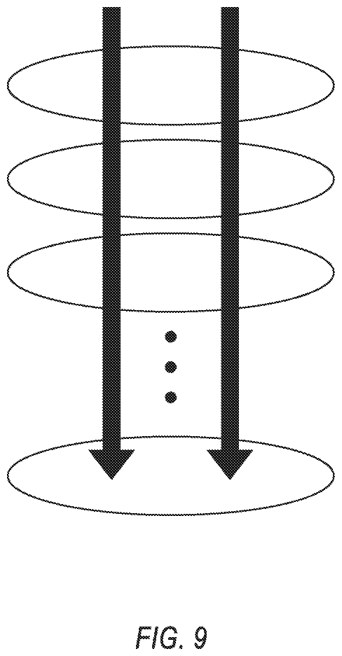

FIG. 9 illustrates the fluence on each layer 116 of the detector 110. This may be the ‘stopping point’ of the proton, e.g., at which layer 116 of the detector 110 the proton was last detected. This stopping point may be used to reconstruct the residual energy of each sub-spot.

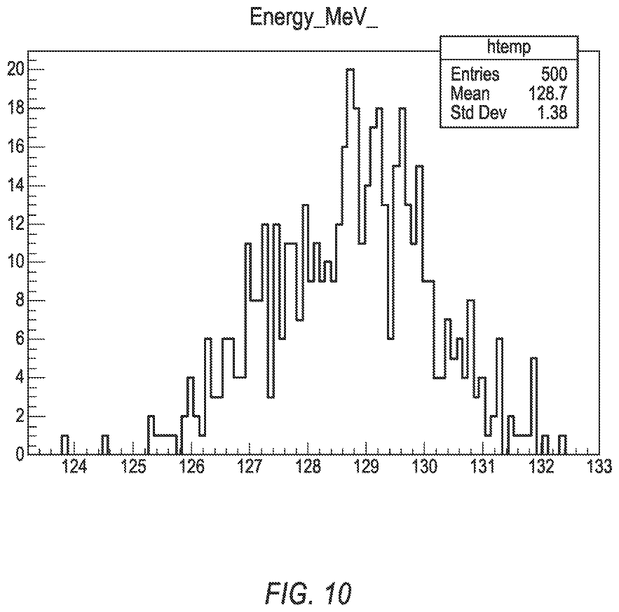

FIG. 10 illustrates an example spectrum of proton residual energy on each sub-spot. Each sub-spot may be reconstructed from accumulating the deposited total energy on each layer 116. Further, the mean entrance angle for protons in each sub-spot may be derived as well.

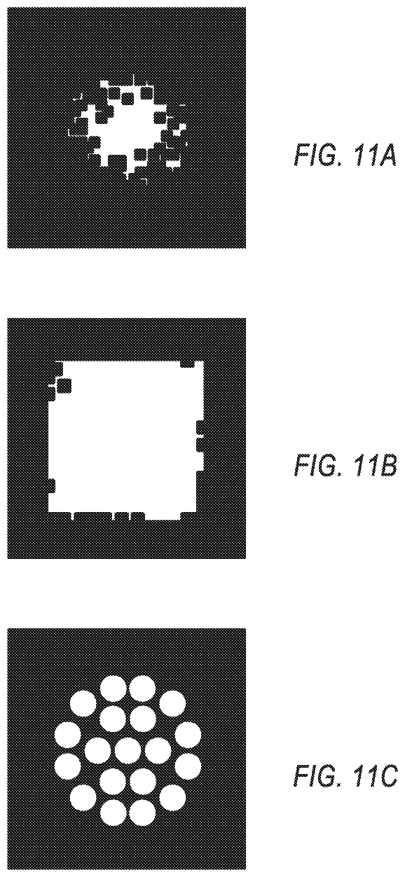

FIG. 11a illustrates an example image where a proton spot with lower number of protons are detected by the detector 110, for example, 500 protons.

FIG. 11b illustrates an example image where a medium level of protons are detected by the detector 110, for example, 5000 protons.

FIG. 11c illustrates an example image of a reconstructed residual energy on the sub-spots. FIG. 11c illustrates a grouping of pixels by applying FIG. 8 to FIG. 11b. Each sub-spot of FIG. 8 may be statistically analyzed to reconstruct the residual energy. For example, each sub-spot has a stopping point, as illustrated in FIG. 11b. Some protons may stop at a first layer of the detector 110. Some (and most) may stop at a third layer. These stopping points may be used to generate the residual energy of FIG. 11c.

When comparing the above method to passive scattering proton beam based pCT, pencil beam systems has the advantage of modulating the scanning pattern, spot size, spot current, spot energy, etc. This sequence, as explained above, is generated by the controller 120 based on pre-knowledge factors such that the residual energy spectrum and fluence of the residual (exit) proton spots are optimized for the detector 110. That is, only certain beams will reach certain layers 115 of the detector 110.

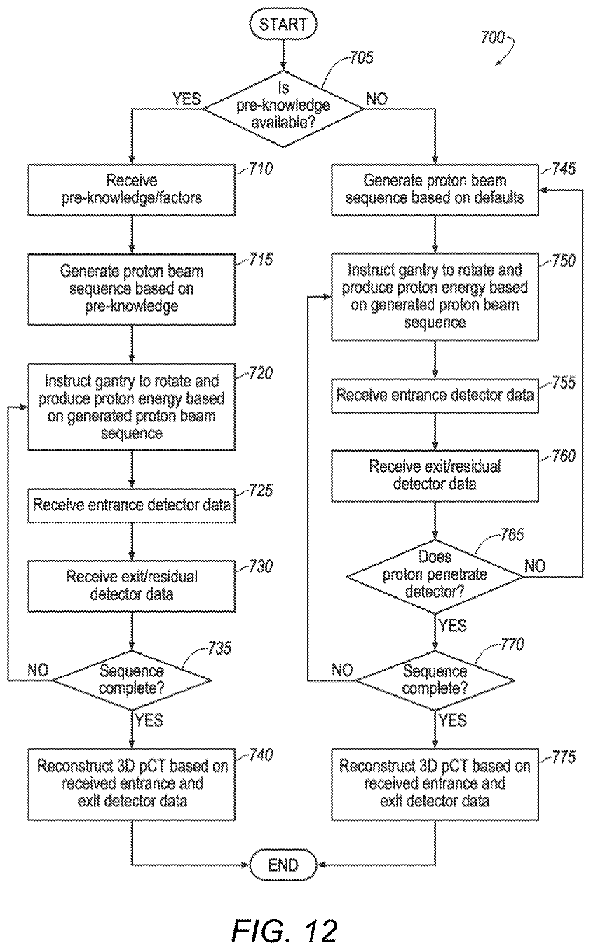

FIG. 12 illustrates an example process 700 for the on-board pCT system 100. The process 700 begins at block 705 where the controller 120 determines whether pre-knowledge or factors relevant to the current patient are available. This may be accomplished by querying the memory 122 for a certain patient number, name, etc. If pre-knowledge is available, the process 700 may proceed to block 710. If not, the process 700 proceeds to block 745.

At block 710, the controller 120 may receive the pre-knowledge from the memory 122. As explained above, the pre-knowledge may be factors or data previously acquired from a patient's MRI, CT, PET, ultrasound, etc.

At block 715, the controller 120 may generate the proton beam sequence based on the pre-knowledge. The sequence may include the energy, position, fluoresce, etc. of the pCT imaging during the gantry continuous rotation.

At block 720, the controller 120 may instruct the gantry to rotate and produce particle beams 118 according to the sequence.

At block 725, the controller 120 may receive entrance detector data from the ion chamber 142 in the nozzle 114 of the gantry 104.

At block 730, the controller 120 may receive exit/residual detector data of the residual particle beams 119 from the multiplayer pixelated residual energy detector 110.

At block 735, the controller 120 may determine whether the sequence is complete. That is, has the gantry completed each rotational angle and produced the particle beams accordingly. If so, the process 700 proceeds to block 740. If not, the process 700 proceeds back to block 720.

At block 740, the controller 120 may reconstruct the pCT image based on the received detector data at blocks 725 and 730.

At block 745, the controller 120 may generate a rough spot energy, scanning sequence, position and fluence for the pCT scanning in response to the pre-knowledge being unavailable. This “preset” or default sequence may permit a starting point for the scanning and create an iterative approach to generate the appropriate proton beam energy when patient data is unknown.

At block 750, the controller 120, similar to block 720, may instruct the gantry to proceed with the default sequence.

At block 755, the controller 120, similar to block 725, may receive entrance detector data.

At block 760, similar to block 730, the controller 120 may receive residual detector data.

At block 765, the controller 120 may determine whether the proton beam penetrated the detector 110. That is, did the proton beam extend from the nozzle 114, through the object/patient, and hit one of the layers 115 of the energy detector 110. If so, the process 700 proceeds to block 770. If not, the process 700 proceeds back to block 745 where the energy of the proton beam is adjusted in order to achieve desirable data at the detector 110 for image reconstruction.

At block 770, the controller 120, similar to block 735, may determine whether the sequence is complete. If so, the process 700 proceeds to block 775. If not, the process 700 returns to block 750.

At block 775, similar to block 740, the controller 120 reconstructs the image based on the detector data.

The process 700 then ends.

Computing devices described herein generally include computer-executable instructions, where the instructions may be executable by one or more computing or hardware devices such as those listed above. Computer-executable instructions may be compiled or interpreted from computer programs created using a variety of programming languages and/or technologies, including, without limitation, and either alone or in combination, Java™, C, C++, Visual Basic, Java Script, Perl, etc. In general, a processor (e.g., a microprocessor) receives instructions, e.g., from a memory, a computer-readable medium, etc., and executes these instructions, thereby performing one or more processes, including one or more of the processes described herein. Such instructions and other data may be stored and transmitted using a variety of computer-readable media.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

1. An on-board proton imaging system, comprising a continuous rotation gantry configured to generate proton beams during rotation thereof to penetrate a patient object;
a beam detector arranged opposite of the gantry around the object and configured to receive residual proton beams having passed through the object;
a controller in communication with the gantry and a multilayer detector, configured to:
instruct the gantry to generate the proton beams based on patient factors;
receive data from the detector indicating at least an energy level of the residual beams; and
generate a three-dimensional image based on the received data.

2. The system of claim 1, wherein the beam detector includes a plurality of layers configured to receive the residual proton beams, wherein each layer is associated with an energy level and receipt of the proton beam at one of the layers indicates an energy level of the residual proton beam.

3. The system of claim 2, wherein the controller is further configured to receive the energy level and a location of at least one of the residual beams from the beam detector.

4. The system of claim 1, wherein the gantry includes a nozzle configured to continuously emit and direct the proton beams while the gantry is continuously rotating.

5. The system of claim 1, wherein the gantry is configured to generate the proton beams at varying energy levels.

6. The system of claim 1, wherein the gantry includes an ionization chamber configured to detect proton beam data including at least one of a fluoresce, position or direction of the proton beam.

7. The system of claim 6, wherein the gantry includes at least one pair of scanning magnets configured to provide the proton beam to the ionization chamber.

8. The system of claim 6, wherein the controller is further configured to generate a three-dimensional image based at least in part on the proton beam data.

9. The system of claim 1, wherein the detector is a ring-like shape configured to surround, at least in part, the object.

10. A proton imaging system, comprising
a memory configured to store patient factors;
a controller in communication with the memory and configured to:
instruct a continuous rotation gantry to generate proton beams based on the patient factors to penetrate a patient object;
receive proton beam data from a beam detector indicating at least an energy level of residual beams having passed through the object; and
generate a three-dimensional image based on the received data.

11. The system of claim 10, wherein the controller is further configured to receive the energy level and a location of at least one of the residual beams from the beam detector.

12. The system of claim 10, wherein the proton beams are generated at varying rotating positions around the object.

13. The system of claim 10, wherein the gantry is configured to generate the proton beams varying energy levels.

14. The system of claim 10, wherein the controller is further configured to generate a three-dimensional image based at least in part on the proton beam data.

15. The system of claim 10, wherein the patient factors are acquired from images of the patient object.
